(19) United States
(12) Patent Application Publication
(10) Pub. No.: US 2006/0173293 A1
(43) Pub. Date: Aug. 3, 2006

(54) Method and apparatus for computer assistance with intramedullary nail procedure

(76) Inventors: Joel Marquart, Pembroke Pines, FL (US); Louis K Arata, Mentor, OH (US); Randall Hand, Clinton, MS (US); Arthur E. Quaid III, North Miami, FL (US); Rony A. Abovitz, Hollywood, FL (US); David Dybala, Cedar Knolls, NJ (US); Robert J. Brumback, Glyndon, MD (US); Ryan Schoenefeld, Ft. Wayne, IN (US)

Correspondence Address: MUNSCH, HARDT, KOPF & HARR, P.C.;INTELLECTUAL PROPERTY DOCKET CLERK, 3800 LINCOLN PLAZA, 500N AKARD STREET, DALLAS, TX, 75201, US

(21) Appl. No.: 11/201,741

(22) Filed: Aug. 11, 2005

Related U.S. Application Data
(63) Continuation of application No. 11/006,513, filed on Dec. 6, 2004.
Application No. 11/006,513 is a continuation of application No. 10/771,851, filed on Feb. 4, 2004.
(60) Provisional application No. 60/445,001, filed on Feb. 4, 2003.

(51) Int. Cl. A61B 5/05

(52) U.S. Cl. 600/426; 600/407

(57) ABSTRACT

A specially-programmed, computer-assisted surgery system is used to reduce the number of fluoroscopic images required to be taken during the course of a intramedullary nail procedure, eliminates the need for a Steinman pin, and assists the surgeon in properly aligning and securing the nail during insertion.

This patent application is a continuation of patent application Ser. No. 11/006,513, entitled “Method and Apparatus for Computer Assistance with Intramedullary Nail Procedure, filed Dec. 6, 2004, which is a continuation of patent application Ser. No. 10/771,851, entitled “Method and Apparatus for Computer Assistance With Intramedullary Nail Procedure,” filed Feb. 4, 2004; and claims the benefit of U.S. provisional patent application Ser. No. 60/445,001, entitled “Method and Apparatus for Computer Assistance With Intramedullary Nail Procedure”, filed Feb. 4, 2003, the disclosure of which is incorporated herein by reference. This application relates to the following U.S. provisional patent applications Ser. No. 60/444,824, entitled “Interactive Computer-Assisted Surgery System and Method”; Ser. No. 60/444,975, entitled “System and Method for Providing Computer Assistance With Spinal Fixation Procedures”; Ser. No. 60/445,078, entitled “Computer-Assisted Knee Replacement Apparatus and Method”; Ser. No. 60/444,989, entitled “Computer-Assisted External Fixation Apparatus and Method”; Ser. No. 60/444,988, entitled “Computer-Assisted Knee Replacement Apparatus and Method”; Ser. No. 60/445,202, entitled “Method and Apparatus for Computer Assistance With Total Hip Replacement Procedure”; and Ser. No. 60/319,924, entitled “Portable, Low-Profile Integrated Computer, Screen and Keyboard for Computer Surgery Applications”; each of which was filed on Feb. 4, 2003 and is incorporated herein by reference. This application also relates to the following applications: U.S. patent application Ser. No. 10/772,083, entitled “Interactive Computer-Assisted Surgery System and Method”; U.S. patent application Ser. No. 10/771,850, entitled “System and Method for Providing Computer Assistance With Spinal Fixation Procedures”; U.S. patent application Ser. No. 10/772,139, entitled “Computer-Assisted Knee Replacement Apparatus and Method”; U.S. patent application Ser. No. 10/772,142, entitled Computer-Assisted External Fixation Apparatus and Method”; U.S. patent application Ser. No. 10/772,085, entitled “Computer-Assisted Knee Replacement Apparatus and Method”; U.S. patent application Ser. No. 10/772,092, entitled “Method and Apparatus for Computer Assistance With Total Hip Replacement Procedure”; and U.S. patent application Ser. No. 10/772,137, entitled “Portable Low-Profile Integrated Computer, Screen and Keyboard for Computer Surgery Applications”; each of which was filed on Feb. 4, 2004 and is incorporated herein by reference.

### TECHNICAL FIELD OF THE INVENTION

The present invention relates generally to computer-assisted surgery systems and surgical navigation systems.

### BACKGROUND OF THE INVENTION

Image-based surgical navigation systems display the positions of surgical tools with respect to preoperative (prior to surgery) or intraoperative (during surgery) image data sets. Two and three dimensional image data sets are used, as well as time-variant images data (i.e. multiple data sets taken at different times). Types of data sets that are primarily used include two-dimensional fluoroscopic images and three-dimensional data sets include magnetic resonance imaging (MRI) scans, computer tomography (CT) scans, positron emission tomography (PET) scans, and angiographic data. Intraoperative images are typically fluoroscopic, as a C-arm fluoroscope is relatively easily positioned with respect to patients and does not require that a patient be moved. Other types of imaging modalities require extensive patient movement and thus are typically used only for preoperative and post-operative imaging.

The most popular navigation systems make use of a tracking or localizing system to track tools, instruments and patients during surgery. These systems locate in predefined coordinate space specially recognizable markers that are attached or affixed to, or possibly inherently a part of, an object such as an instrument or a patient. Markers can take several forms, including those that can be located using optical (or visual), electromagnetic, radio or acoustic methods. Furthermore, at least in the case of optical or visual systems, location of an object's position may be based on intrinsic features or landmarks that, in effect, function as recognizable markers. Markers will have a known, geometrical arrangement with respect to, typically, an end point and/or axis of the instrument. Thus, objects can be recognized at least in part from the geometry of the markers (assuming that the geometry is unique), and the orientation of the axis and location of endpoint within a frame of reference deduced from the positions of the markers.

Present-day tracking systems are typically optical, functioning primarily in the infrared range. They usually include a stationary stereo camera pair that is focused around the area of interest and sensitive to infrared radiation. Markers emit infrared radiation, either actively or passively. An example of an active marker is light-emitting diodes (LEDs). An example of a passive marker is a reflective marker, such as ball-shaped marker with a surface that reflects incident infrared radiation. Passive systems require a an infrared radiation source to illuminate the area of focus. A magnetic system may have a stationary field generator that emits a magnetic field that is sensed by small coils integrated into the tracked tools.

Most CAS systems are capable of continuously tracking, in effect, the position of tools (sometimes also called instruments). With knowledge of the position of the relationship between the tool and the patient and the patient and image data sets, a system is able to continually superimpose a representation of the tool on the image in the same relationship to the anatomy in the image as the relationship of the actual tool to the patient's anatomy. To obtain these relationships, the coordinate system of the image data set must be registered to the relevant anatomy of the actual patient portions of the of the patient's anatomy in the coordinate system of the tracking system. There are several known registration methods.

In CAS systems that are capable of using two-dimensional image data sets, multiple images are usually taken from different angles and registered to each other so that a representation of the tool or other object (which can be real or virtual) can be, in effect, projected into each image. As the position of the object changes in three dimensional space, its projection into each image is simultaneously updated. In order to register two or more two-dimensional data images together, the images are acquired with what is called a registration phantom in the field of view of the image device. In the case of a two dimensional fluoroscopic images, the phantom is a radio-translucent body holding radio-opaque fiducials having a known geometric relationship. Knowing the actual position of the fiducials in three dimensional space when each of the images are taken permits determination of a relationship between the position of the fiducials and their respective shadows in each of the images. This relationship can then be used to create a transform for mapping between points in three-dimensional space and each of the images. By knowing the positions of the fiducials with respect to the tracking system's frame of reference, the relative positions of tracked tools with respect to the patient's anatomy can be accurately indicated in each of the images, presuming the patient does not move after the image is acquired, or that the relevant are portions of the patient's anatomy is are tracked. A more detailed explanation of registration of fluoroscopic images and coordination of representations of objects in patient space superimposed in the images is found in U.S. Pat. No. 6,198,794 of Peshkin, et al., entitled “Apparatus and method for planning a stereotactic surgical procedure using coordinated fluoroscopy”.

### SUMMARY OF THE INVENTION

The invention is generally directed to improved computer-implemented methods and apparatus for further reducing the invasiveness of surgical procedures, eliminating or reducing the need for external fixtures in certain surgical procedures, and/or improving the precision and/or consistency of surgical procedures. The invention finds particular advantage in orthopedic procedures involving implantation of devices, though it may also be used in connection with other types of surgical procedures.

For example, a surgeon encounters or has to overcome several problems during insertion of an intramedullary nail (“IM nail”), an elongated rod-shaped prosthetic device, into the canal of a fractured femur. These problems include matching the leg length of the injured leg with the well leg of the patient, improper rotation of the injured leg, and unpredictable flexing of the distal end of the nail. To reduce the incidence of malrotation of the leg, fluoroscopic images are taken frequently during the procedure, thus exposing the patient and operating room personnel to radiation. Furthermore, implantation of the IM nail using traditional methods requires use of an extra pin for determining the version of the leg for proper alignment of the rod, as well as use of a special, radio-translucent drill so that fluoroscopic images can be captured during insertion of screws into the distal end of the femur to secure the distal end of the nail.

To address one or more of these problems, various aspects of a specially-programmed, computer-assisted surgery system are used to reduce the number of fluoroscopic images required to be taken, especially during the course of the procedure, eliminate the need for a Steinman pin, and assist the surgeon in properly aligning and securing the nail during insertion. A preferred embodiment of such an application for programming a computer-assisted surgery system is described below.

### BRIEF DESCRIPTION OF THE DRAWINGS

For a more complete understanding of the present invention, the objects and advantages thereof, reference is now made to the following descriptions taken in connection with the accompanying drawings in which:

FIG. 1 is a block diagram of an exemplary computer-assisted surgery system;

FIG. 2 is a simple diagram of a patient having a fractured femur and prepared for surgery;

FIG. 3 is a flow chart of basic steps of an application program for assisting with or guiding the planning and execution of a surgical procedure and navigation during the procedure;

FIG. 4 is a flow chart of basic set-up steps for an application for assisting with planning of, and navigation during, an intramedullary nail procedure;

FIG. 5 is a flow chart of basic steps of a reference determination portion of the planning phase of the application of FIG. 4;

FIG. 6A is a more detailed flow chart of basic steps of reference dimensions and a nail determination portion of a phase of the application of FIG. 4;

FIG. 6B is a more detailed flow chart of planning injured leg for determination of fracture site, length and anteversion for the application of FIG. 4;

FIG. 7 is a detailed flow chart of a navigation/execution phase of the application of FIG. 4; and

FIGS. 8-27 are representative screens of graphical user interface pages displayed by the computer-assisted surgery system of FIG. 1 during use of the application of FIG. 4.

### DETAILED DESCRIPTION OF THE DRAWINGS

In the following description, like numbers refer to like elements. References to “surgeon” include any user of a computer-assisted surgical system, a surgeon being typically a primary user.

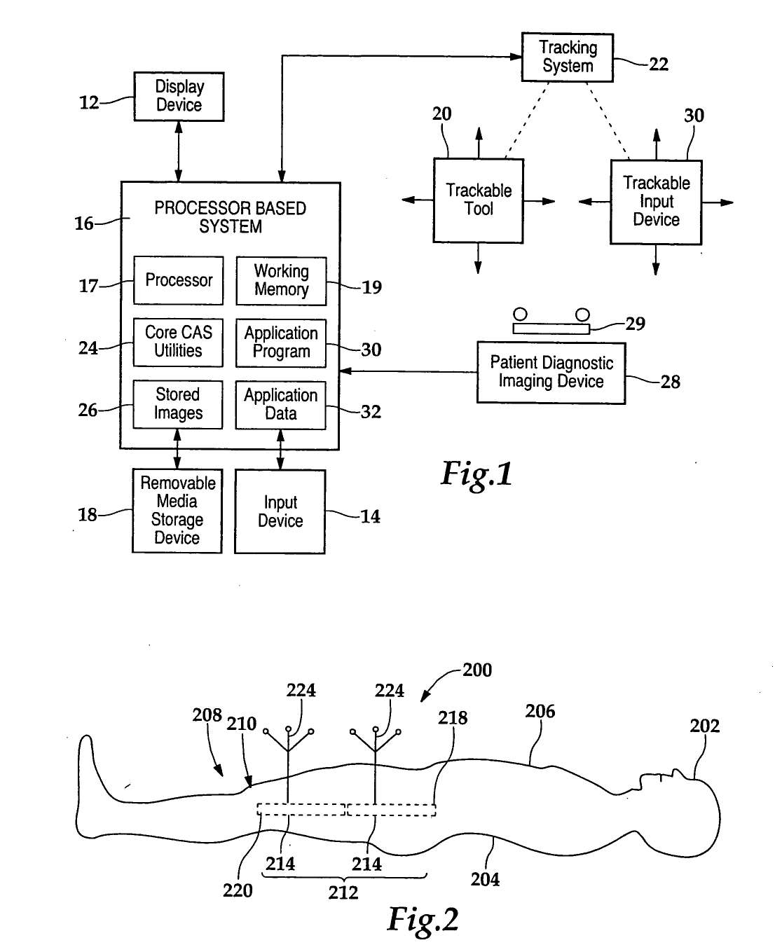

FIG. 1 is a block diagram of an exemplary computer-assisted surgery (CAS) system 10. Computer-assisted surgery system (CAS) 10 comprises a display device 12, an input device 14, and a processor-based system 16, for example, a computer. Display device 12 may be any display device now known or later developed for displaying two-dimensional and/or three-dimensional diagnostic images, for example, a monitor, a touch screen, a wearable display, a projection display, a head-mounted display, stereoscopic views, a holographic display, a display device capable of displaying image(s) projected from an image projecting device, for example, a projector, and/or the like. Input device 14 may be any input device now known or later developed, for example, a keyboard, a mouse, a trackball, a trackable probe and/or the like. The processor-based system is preferably programmable and includes one or more processors 16a, working memory 16b for temporary program and data storage that will be used primarily by the processor, and storage for programs and data, preferably persistent, such as a disk drive. Removable media storage device 18 can also be used to store programs and/or transfer to or from the transfer programs.

Tracking system 22 continuously determines, or tracks, the position of one or more trackable markers disposed on, incorporated into, or inherently a part of surgical tools or instruments 20 with respect to a three-dimensional coordinate frame of reference. With information from the tracking system on the location of the trackable markers, CAS system 10 is programmed to be able to determine the three-dimensional coordinates of an endpoint or tip of a tool and, optionally, its primary axis using predefined or known (e.g. from calibration) geometrical relationships between trackable markers on the tool and the end point and/or axis of the tool. A patient, or portions of the patient's anatomy, can also be tracked by attachment of arrays of trackable markers.

The CAS system can be used for both planning surgical procedures (including planning during surgery) and for navigation. It is therefore preferably programmed with software for providing basic image-guided surgery functions, including those necessary determining the position of the tip and axis of instruments and for registering a patient and preoperative and/or intraoperative diagnostic image data sets to the coordinate system of the tracking system. The programmed instructions for these functions are indicated as core CAS utilities 24. These capabilities allow the relationship of a tracked instrument to a patient to be displayed and constantly updated in real time by the CAS system overlaying a representation of the tracked instrument on one or more graphical images of the patient's internal anatomy on display device 12. The graphical images are constructed from one or more stored image data sets 26 acquired from diagnostic imaging device 28. Imaging device may be a fluoroscope, such as a C-arm fluoroscope, capable of being positioned around a patient lying on an operating table. It may also be a MR, CT or other type of imaging device in the room or permanently located elsewhere. Where more than one image is shown, as when multiple fluoroscopic images are simultaneously displayed of display device 12, the representation of the tracked instrument or tool is coordinated between the different images. However, CAS system can be used in some procedures without the diagnostic image data sets, with only the patient being registered. Thus, the CAS system need not support the use of diagnostic images in some applications—i.e. an imageless application.

Furthermore, as disclosed herein, the CAS system may be used to run application-specific programs 30 that are directed to assisting a surgeon with planning and/or navigation during specific types of procedures. For example, the application programs may display predefined pages or images corresponding to specific steps or stages of a surgical procedure. At a particular stage or part of a program, a surgeon may be automatically prompted to perform certain tasks or to define or enter specific data that will permit, for example, the program to determine and display appropriate placement and alignment of instrumentation or implants or provide feedback to the surgeon. Other pages may be set up to display diagnostic images for navigation and to provide certain data that is calculated by the system for feedback to the surgeon. Instead of or in addition to using visual means, the CAS system could also communicate information in ways, including using audibly (e.g. using voice synthesis) and tactilely, such as by using a haptic interface of device. For example, in addition to indicating visually a trajectory for a drill or saw on the screen, a CAS system may feedback to a surgeon information whether he is nearing some object or is on course with a audible sound or by application of a force or other tactile sensation to the surgeon's hand.

To further reduce the burden on the surgeon, the program may automatically detect the stage of the procedure by recognizing/identifying the instrument picked up by a surgeon and move immediately to the part of the program in which that tool is used. Application data 32—data generated or used by the application—may also be stored processor-based system.

Various types of user input methods can be used to improve ease of use of the CAS system during surgery. One example is the use of speech recognition to permit a doctor to speak a command. Another example is the use of a tracked object to sense a gesture by a surgeon, which is interpreted as an input to the CAS system. The meaning of the gesture could further depend on the state of the CAS system or the current step in an application process executing on the CAS system. Again, as an example, a gesture may instruct the CAS system to capture the current position of the object. One way of detecting a gesture is to occlude temporarily one or more of the trackable markers on the tracked object (e.g. a probe) for a period of time, causing loss of the CAS system's ability to track the object. A temporary visual occlusion of a certain length (or within a certain range of time), coupled with the tracked object being in the same position before the occlusion and after the occlusion, would be interpreted as an input gesture. A visual or audible indicator that a gesture has been recognized could be used to provide feedback to the surgeon.

Yet another example of such an input method is the use of tracking system 22 in combination with one or more trackable data input devices 34. Defined with respect to the trackable input device 34 are one or more defined input areas, which can be two-dimensional or three-dimensional. These defined input areas are visually indicated on the trackable input device so that a surgeon can see them. For example, the input areas may be visually defined on an object by representations of buttons, numbers, letters, words, slides and/or other conventional input devices. The geometric relationship between each defined input area and the trackable input device is known and stored in processor-based system 16. Thus, the processor can determine when another trackable object touches or is in close proximity a defined input area and recognize it as an indication of a user input to the processor-based systems. For example, when a tip of a tracked pointer is brought into close proximity to one of the defined input areas, the processor-based system will recognize the tool near the defined input area and treat it as a user input associated with that defined input area. Preferably, representations on the trackable user input correspond with user input selections (e.g. buttons) on a graphical user interface on display device 12. The trackable input device may be formed on the surface of any type of trackable device, including devices used for other purposes. In a preferred embodiment, representations of user input functions for graphical user interface are visually defined on a rear, flat surface of a base of a tool calibrator.

Processor-based system 16 is, in one example, a programmable computer that is programmed to execute only when single-use or multiple-use software is loaded from, for example, removable media. The software would include, for example the application program 30 for use with a specific type of procedure. Media storing the application program can be sold bundled with disposable instruments specifically intended for the procedure. The application program would be loaded into the processor-based system and stored there for use during one (or a defined number) of procedures before being disabled. Thus, the application program need not be distributed with the CAS system. Furthermore, application programs can be designed to work with specific tools and implants and distributed with those tools and implants. Preferably, also, the most current core CAS utilities may also be stored with the application program. If the core CAS utilities on the processor-based system are outdated, they can be replaced with the most current utilities.

FIG. 2 is intended to be a representative patient with a representative fractured femur. The representative patient 200, represented by a head 202, torso 204, arm 206, leg 208 and knee 210. Indicated by dashed lines in the upper leg, above the knee, is a femur 212 that is fractured and separated into two pieces, which will be referred to as the proximal fragment 214 and distal fragment 216 to correspond with the proximal end 218 and distal end 220 of the femur. A trackable marker array 212, which can be tracked by the CAS system 10 (FIG. 1), is attached to, respectively, the proximal piece 214 and distal piece 216 of the femur so that the relative position of the two pieces can be tracked during implantation of an IM nail into the femur.

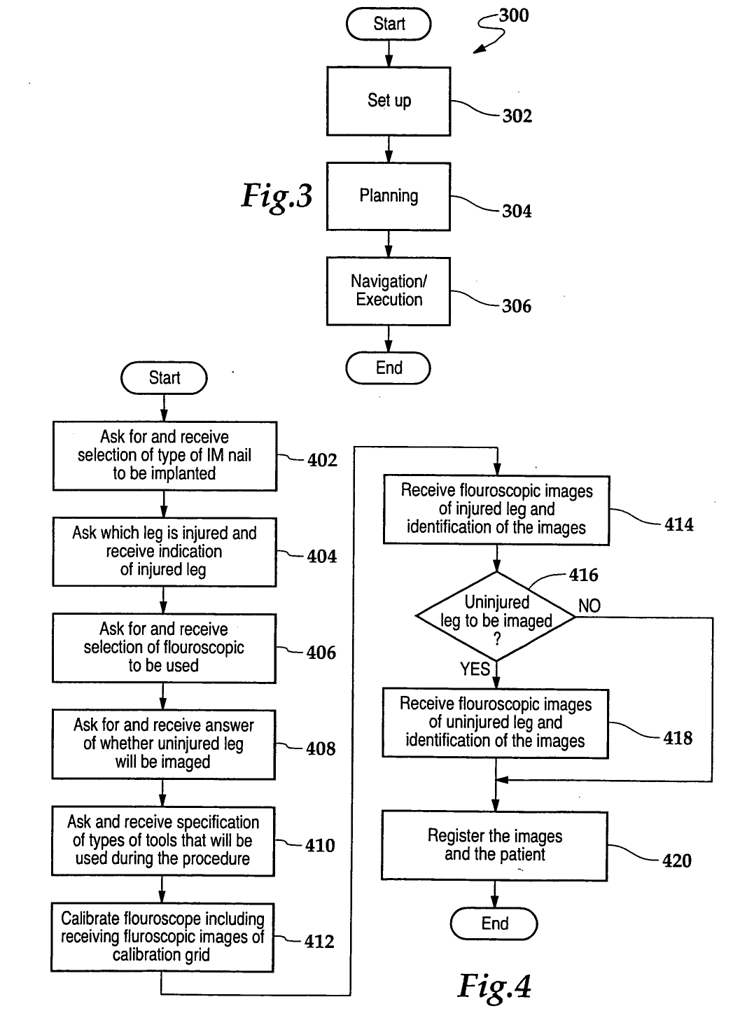

Referring now to FIG. 3, the CAS system assists a surgeon in performing an IM nail implantation by executing a process 300 that has three basic phases: set-up phase 302, planning phase 304 and navigation phase 306. The set-up phase involves the surgeon specifying to the process which type of IM nail to be used which leg is to be operated on, type of fracture, instruments and/or tools to be tracked during the procedure, and model fluoroscope to be used, which leg is to be operated on, type of fracture, instruments, and/or tools during the process and certain other options such as determining to image and plan using the uninjured leg. The set-up phase allows for skipping certain steps during the navigation or execution stage so that it flows more efficiently to the surgeon's preferences or needs. The planning phase involves using fluoroscopic images to gather reference information on leg version (rotation angle) and length from the surgeon and to select nail dimensions, and placement and length of screws used to secure the nail. The navigation or execution stage tracks the surgeon's instruments and trackable markers implanted in or attached to the patient's femur and provides alignment information and feedback on version and length.

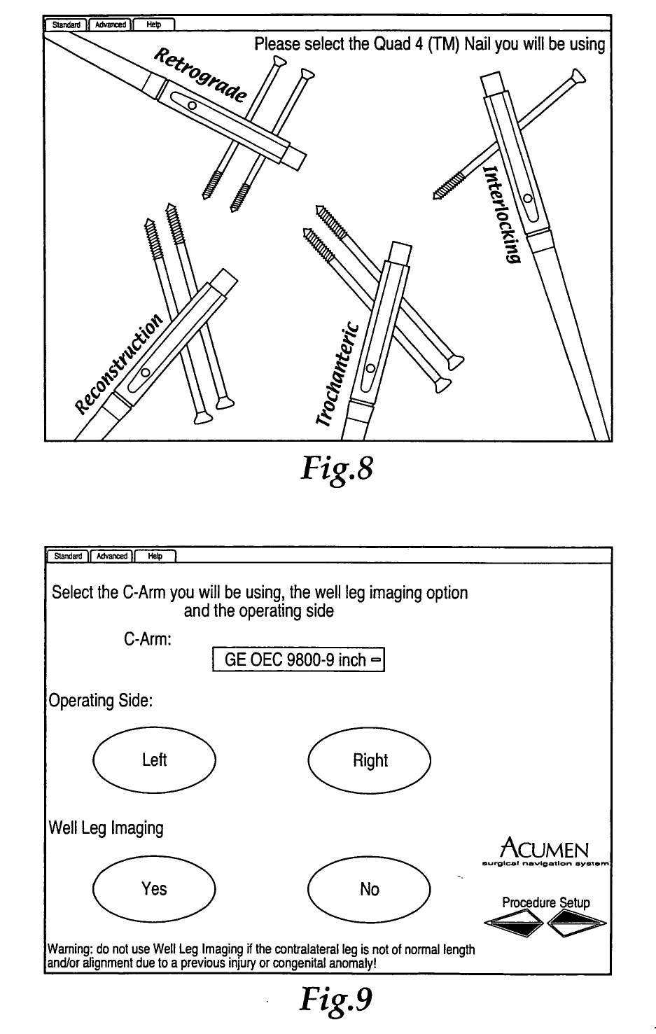

Process 300, or parts thereof, preferably display a series of pages corresponding to stages or sub-procedures, each page being set up to display directions and information (including images) relevant to the stage of the procedure. However, as previously mentioned, the CAS system may in addition to the pages or in place of the pages, communicate some or all of this information by other means, including audible and haptic means. Although the process may constrain what a surgeon does in terms of the ordering of certain steps, the process preferably follows the surgeon, rather than requiring the surgeon to follow the process. This is particularly useful during the planning and navigation or execution phases of the process, where the surgeon may need to go back and change a plan or repeat steps. Thus, in the following explanation of process 300, some steps may be performed out of sequence or repeated. The surgeon may indicate to the process the stage he or she is in or wants to go to. This may be done through user input or by the process automatically recognizing when the surgeon has either finished a stage or is preparing to go to another stage (not necessarily the next stage) by, for example, the surgeon picking up an instrument used in a particular stage and showing it to the cameras of the tracking system. Details of the process 300 will be described with reference to representative examples of screens from such pages, shown in FIGS. 8-27. These screens contemplate use of IM nails for a specific vendor. However, the process and concepts embodied or represented by the pages are not limited to any specific vendor, and aspects thereof may be employed in connection with surgical planning and guidance systems for similar types of implants.

Referring to FIG. 4 and FIGS. 8-13, step 402 asks the surgeon to identify or select which of a plurality of IM nail types or families will be used. This information is used for representing the IM nail on images taken of the patient's leg and providing feedback to the surgeon on the position of the nail during the nail insertion. If the process is set up for only one type of nail, this step may be skipped. FIG. 8 is a screen of a representative example of such a page, in this case showing four families of IM nails from a particular vendor. At steps 404, 406, and 408, the process requests the surgeon to specify which leg is injured, what type of fluoroscope will be used, and whether the uninjured leg of the patient will be used in planning. FIG. 9 is an example of a graphical interface displaying the options for selection by the surgeon. Although the use a fluoroscopic images has certain advantages, other types of images can be used in place of, or in addition to, the fluoroscopic images, including without limitation preoperative three-dimensional data sets such as CT and MRI scans.

At step 410 the surgeon is asked to specify application-specific tools that he will use during the procedure that can be or will be tracked. Surgeons may prefer to use different tools for a given step, and this step permits the surgeon to select the tool of choice so that the CAS system can properly track it. The application may display a different page at a given step, or display pages in a different order, based on selection of the tool. Furthermore, a surgeon may, for example, elect not to use a tool during a given step, or not have it tracked. The process will adjust as necessary to accommodate the preferences to avoid forcing a surgeon to find ways to bypass steps or alter presentation of the pages. The CAS system is typically programmed or set up to operate with a probe and other basic tools that a surgeon may use.

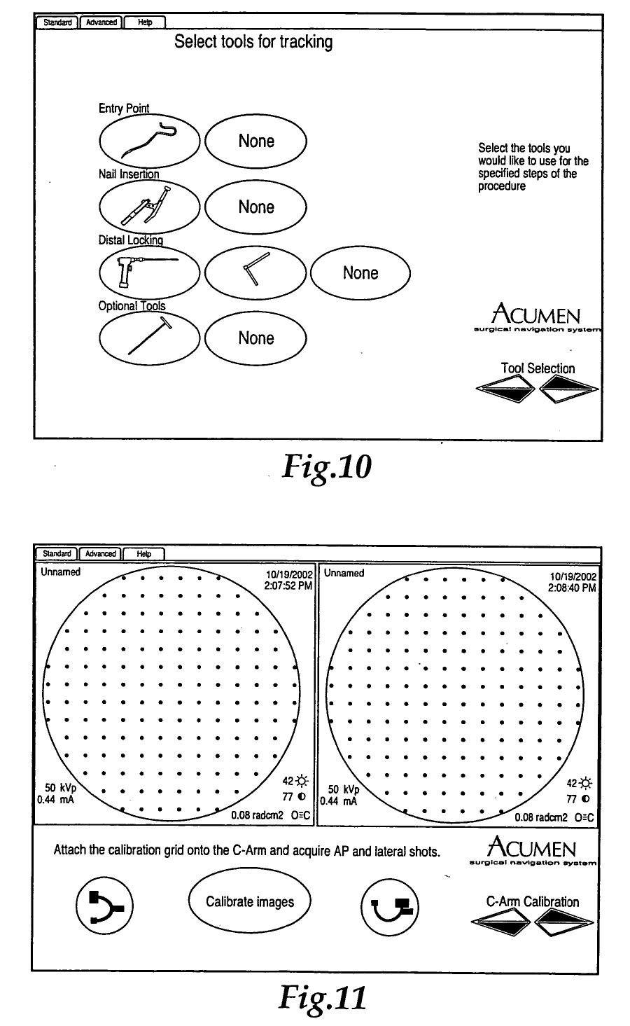

Preferably, the surgeon is given a list of the tool or tools that the application can track, from which he may select. FIG. 10 shows an example of a page that displays the tools that the application is capable of or set up to track for the basic steps of the surgical procedure. The display permits the surgeon to visually select the tool (or not to have a tool) and verify the selection. The tool listing in the illustrated example is also grouped by basic stages of the process. In the example, options are given for the tool that will be used for defining an entry point in the femur prior to insertion of the nail, the tool used for nail insertion, the instrument used for drilling holes to insert screws for locking the distal end of the nail, and other tools that the surgeon may want to use. Thus, in the example, if no tool is selected for specifying the entry point, the program will not expect to receive an indication for the entry point and will not attempt to display the selected point on a diagnostic image. If, for example, a surgeon selects a power drill instead of a hand drill for distal locking, the CAS system will automatically assume that it is tracking a power drill during the distal locking step.

At step 412, the CAS system calibrates the selected fluoroscope using known methods. The interface for this step is illustrated in FIG. 11.

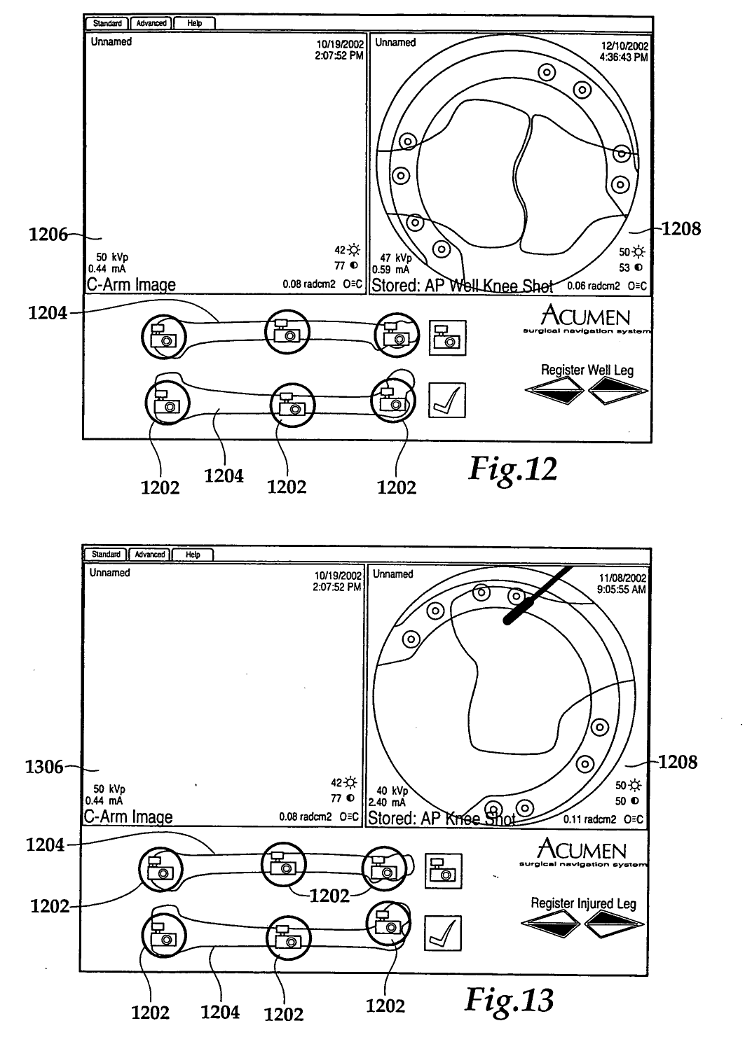

Steps 414, 416, 418 and 420 direct the acquisition of certain fluoroscopic images during the procedure, followed by registration of those images using known methods. If the surgeon specified that the well leg would be used for reference, images of the well leg are acquired in addition to images of the injured leg. Exemplary screen shots of the pages corresponding to the acquisition and registration of the well leg and injured leg are shown in FIGS. 12 and 13, respectively. What images are needed or desirable are listed and identified with respect to the list when they are acquired. In the illustrated examples, the required or desirable images are listed with reference to target areas 1202 defined on diagrams 1204 of a femur. The diagrams show a femur from an anterior/posterior and from a medial/lateral view. It is preferred to acquire an anterior/posterior (A/P) and a medial/lateral (M/L) image of each of the proximal and distal ends of the femur, fluoroscopic image pair is around the midshaft of the femur. Area 1202 may consist of two fracture sites, depending upon the set-up phase. This allows for handling a compound fracture and allows the surgeon to image the proximal and distal fractures in separate shots. Each A/P and M/L fluoroscopic image pair is preferably shown in two, side-by-side windows on the display. During image acquisition, window 1206 displays a current image from the fluoroscope. Once a surgeon is satisfied with an image, it is saved or stored by the CAS system upon appropriate input from the surgeon and is moved to adjacent window 1208 for registration.

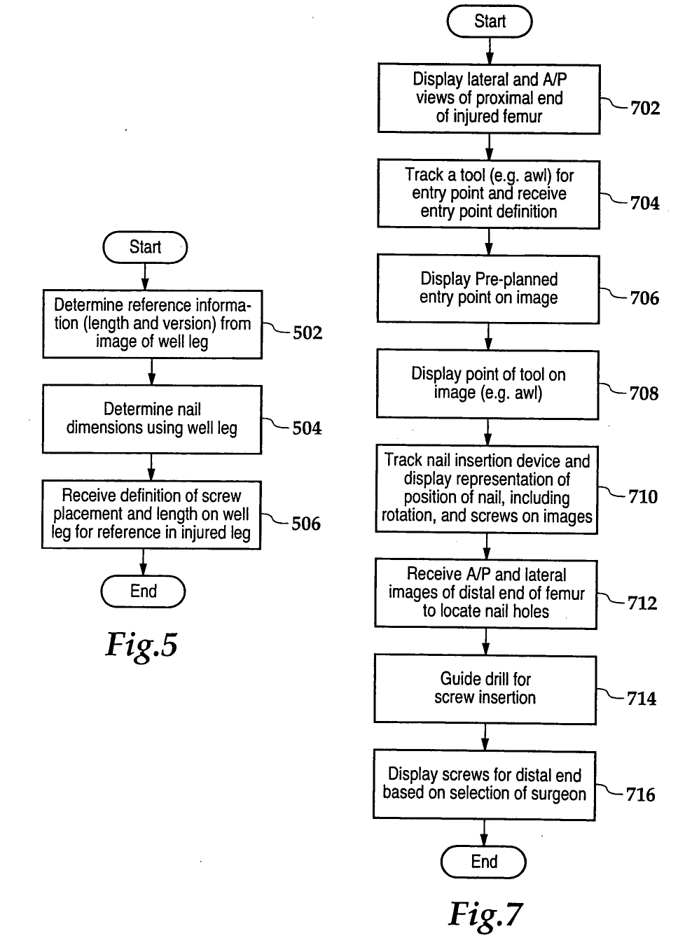

Referring now to FIG. 5, if a well leg is imaged, the planning stage starts with a process 500. With the images of the well leg, certain reference information, namely a reference length and version, are determined at step 502, based on information indicated on the images by the surgeon. Assuming that the injured and well legs are anatomically similar, the well leg may also be used to at least initially determine appropriate nail length and diameter at step 504. Using the well leg to determine this information may be desirable in the event it is difficult to determine this information from the injured leg. As indicated by step 506, screw length placement and length may also be determined.

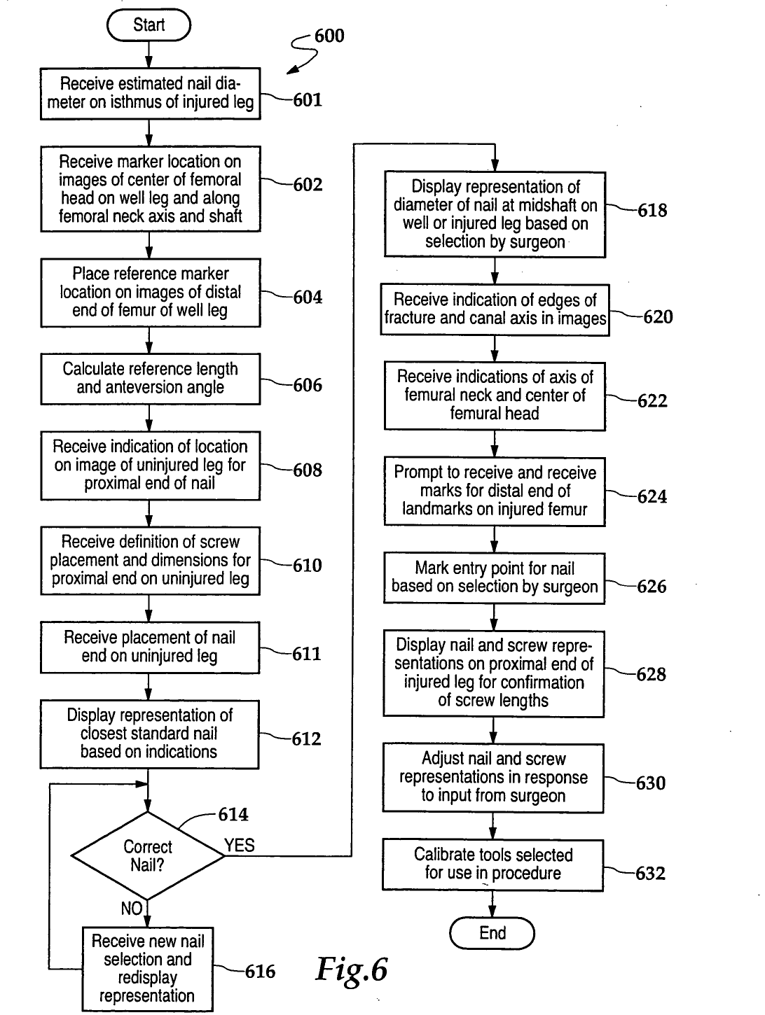

FIGS. 6A and 6B illustrate in the planning stage in greater detail. Steps 602 to 608 involve determination of a reference length and version of the leg or femur. During these steps the surgeon is prompted to indicate in the acquired images of the well leg certain anatomical landmarks, preferably the center of the femoral head, the axis of the femoral neck and shaft, an axis that extends transverse to the condyles at the posterior-most points of the condyles (the trans-epicondylar axis). However, other recognizable landmarks could be used for calculating a reference length and/or version.

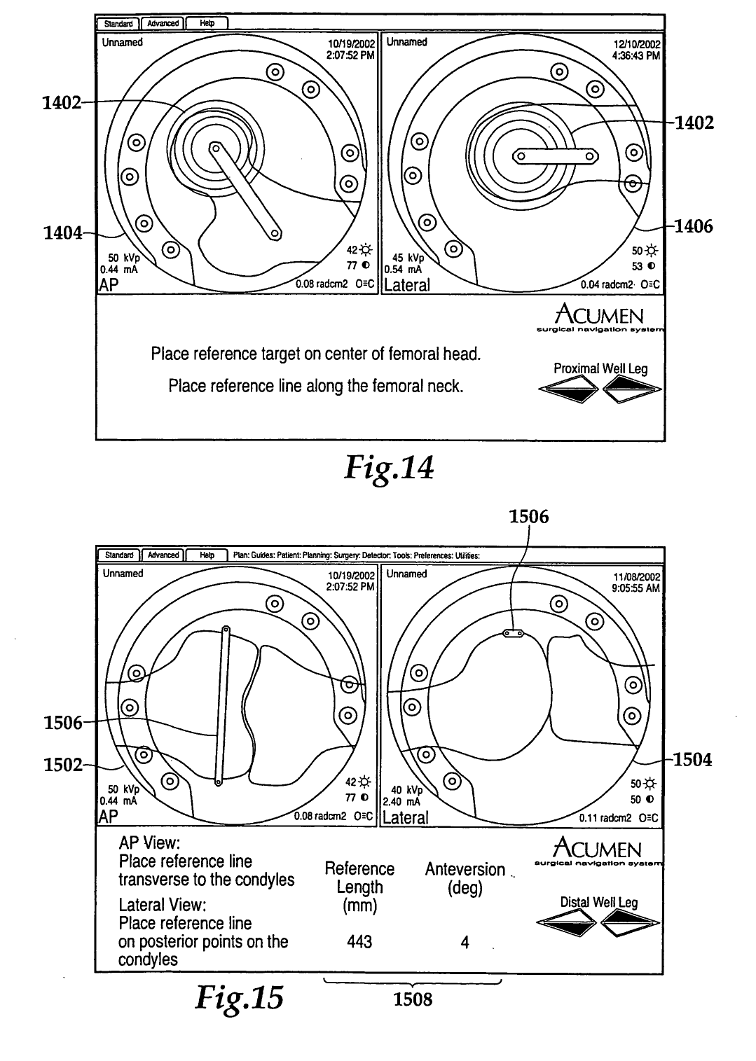

At steps 601 and 602 the surgeon is prompted to indicate, and the process receives, an estimated nail diameter on isthmus of uninjured leg and the center of the femoral head and the axis of its neck with reference to displayed A/P and M/L images of the proximal end of the femur. As illustrated in representative page or interface of FIG. 14, a “bull's-eye” marker 1402 is superimposed on A/P image 1404 and M/L image 1406 for assisting the surgeon in identifying the center of the generally spherical femoral head in both images. This bull's-eye marker is a two-dimensional projection of a series of nested, virtual spheres in the three dimensional space of the patient. As the surgeon moves the marker with respect to one image, its position is automatically updated with respect to the second image. The surgeon is, in effect, moving the virtual spheres. From the center point of the bull's-eye marker to a second end point extends another marker in the form of line 1408. It represents a virtual guide wire in the three-dimensional space of the patient. The surgeon moves this virtual guide wire so that it extends along the axis of the femoral neck. Once the surgeon indicates that markers are in the correct position, the process moves automatically to step 604.

At step 604, the process displays A/P and lateral images of the distal end of the femur. The surgeon indicates on the images a marker for services as a reference point for determining a reference length for the femur. The program stores this information. At step 606, the reference length is calculated using the references marked on the proximal end of the femur and the reference marked on the distal end of the femur. The program also prompts, using, for example, directions displayed on the displayed page, and receives from the surgeon at step 406 the position and orientation for the trans-epicondylar axis of the femur. FIG. 15 is an example of a screen used in these steps. The acquired A/P and lateral images, 1502 and 1504, respectively, of the distal end of the well femur are displayed, along with a reference line 1506. The surgeon manipulates the position and orientation of the reference line in A/P image 1502 so that it is aligned with the trans-epicondylar axis. In the lateral view the surgeon manipulates the reference line 1506 so that it is positioned on the posterior most points of the condyles. Using the definition of the femoral neck axis received at step 604 and the definition of the trans-epicondylar axis received at step 406, a reference version is calculated at step 608. The trans-epicondylar axis also serves as a reference point for calculating a reference length. In order to have meaningful version and reference information, true A/P and lateral views of the distal end of the femur should be acquired, or they should be least taken at the substantially same angles as the A/P and lateral images of the distal end of the injured femur. The reference length and version are stored and displayed in area 1508 of the screen. As an alternative to using a reference line, the version may be calculated using a true lateral image of the distal end and placing a reference point on the knee center in both the A/P and lateral images.

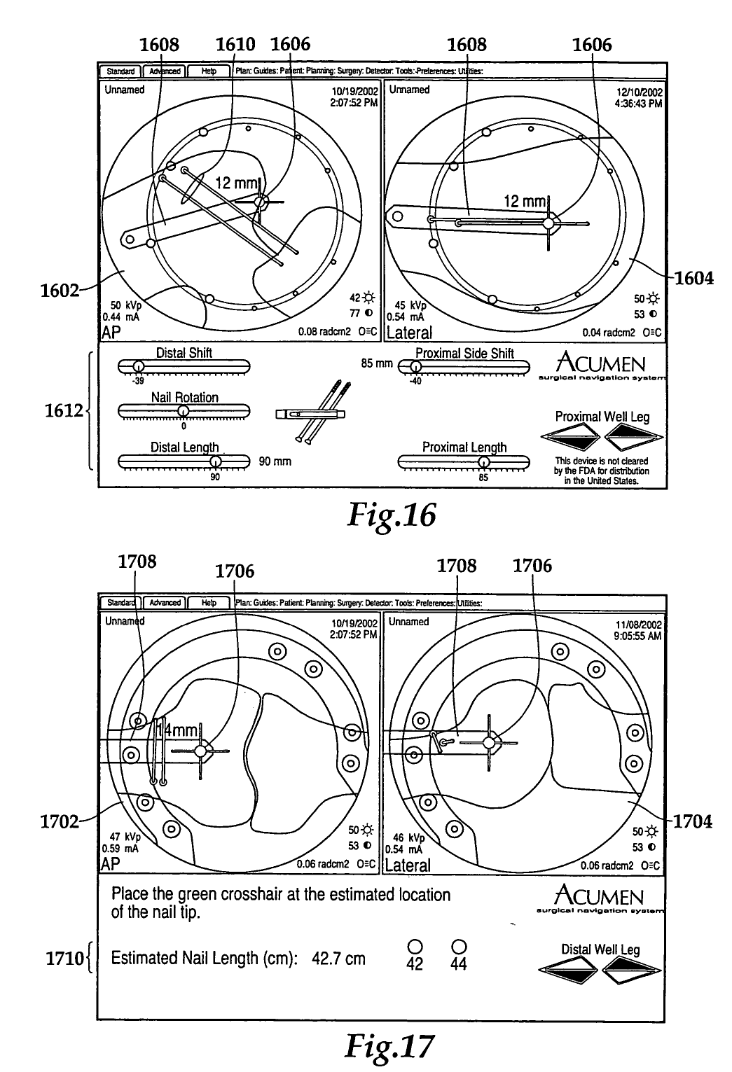

Steps 608, 610, 611, 612, 614 and 616 assist the surgeon with selecting a nail of appropriate length and screw dimensions using the well leg. At step 608, the surgeon indicates, with respect to A/P and M/L images of the distal and proximal ends of the femur, end points for the nail. The process automatically determines the distance between the end points and then it selects and displays on the images a representation of the closest standard length nail. As indicated by steps 610 and 611, screw placement and dimensions for the proximal end of the nail and the placement of the nail end are indicated with respect to the uninjured leg. A representation of the closest standard nail to the indications is then displayed at step 612. The surgeon is then permitted to change, shift, rotate and move the representation in order to check its fit. If the fit is not correct, the surgeon can change the end points and/or select a different nail, as indicated by steps 614 and 616. FIG. 16 is a representative screen from an example of a user interface page displayed on the CAS system implementing these steps. The page includes the stored A/P image 1602 and M/L image 1604 of the proximal end of the well-leg femur. Superimposed on this image is a marker, in the form of a cross-hair graphic 1606, for marking the estimated proximal end of the nail that will be implanted in the other (injured) leg. A representation 1608 of the proximal end of the nail is also preferably superimposed on the two images, along with representations 1610 of screws that will be inserted through the proximal end of the femur and nail once the nail is fully inserted. The surgeon is permitted to change, shift, rotate and move the representation of the screws in order to check its fit. The page includes inputs for changing the position of the cross-hairs and representations. FIG. 17 is a representative screen of a page for the surgeon to mark an estimated location for the tip of the nail. The page includes the stored A/P image 1602 and M/L image 1606 of the distal end of the well- leg femur. It prompts the surgeon to move a marker, namely, the cross-hairs graphic 1706, to the estimated tip of the nail. The program then provides an estimated nail length and displays the two closest standard lengths for the type of nail being used on line 1710. The surgeon selects the desired length and the program moves the nail representation to the correct length for the surgeon to confirm that selected length.

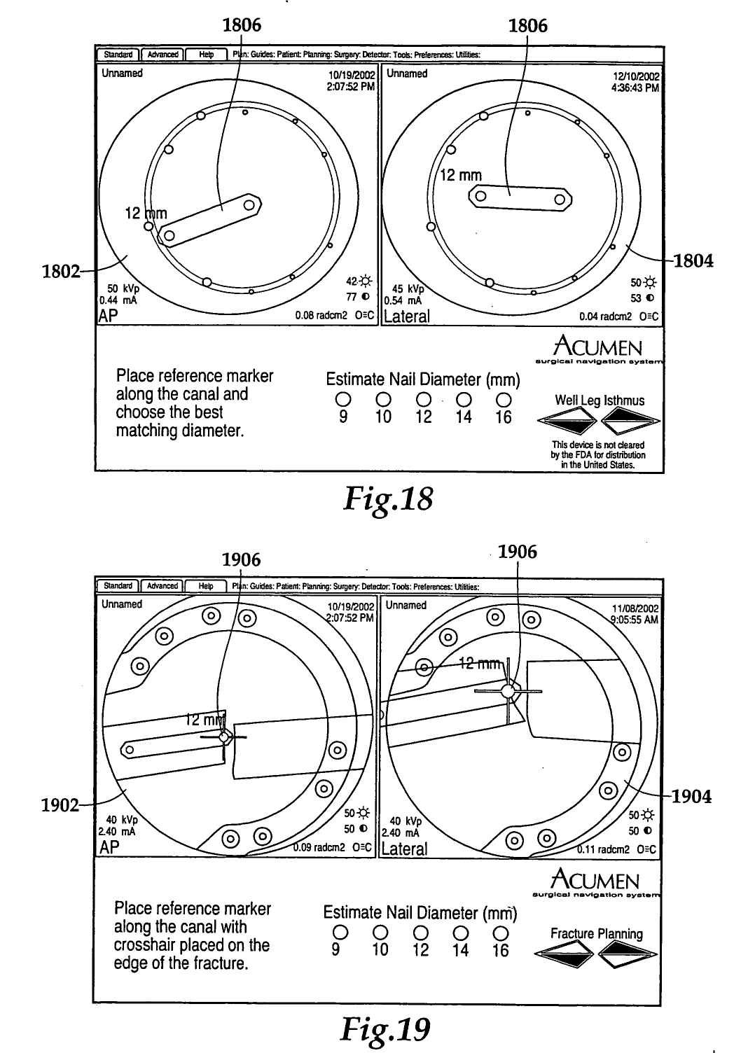

Referring now to FIG. 6B, the diameter of the nail is estimated using at step 618, the midshaft (isthmus) of the well or injured leg. FIG. 18 assumes that well-leg images were acquired at the midshaft of that leg's femur. The diameter of the canal of the femur, through which the nail will be inserted, is its narrowest at the isthmus. The page instructs the surgeon to place a reference marker 1806 along the canal of the femur and then select the best matching diameter from a list. As different nail diameters are selected, the width of the reference marker, which is a projection of a virtual, cylindrical object (corresponding generally to a diameter of a nail) in the three-dimensional patient space into the two-dimensional fluoroscopic images, changes. Once the surgeon decides on a diameter, it is stored and the process moves to injured leg planning.

At step 620, the surgeon is prompted to mark in the images showing the edge of the fracture at the canal of the femur. A representative screen of the page displayed for this step is shown in FIG. 19. The stored A/P image 1902 and the stored lateral MIL image 1904 of the midshaft of the injured femur is displayed, and cross-hairs marker 1906 is also displayed and can be moved by the surgeon to mark the edge of the fracture. A representation 1908 of the nail is also superimposed for the surgeon to check and, if necessary, change the estimated nail diameter that will fit through the canal at the point of fracture.

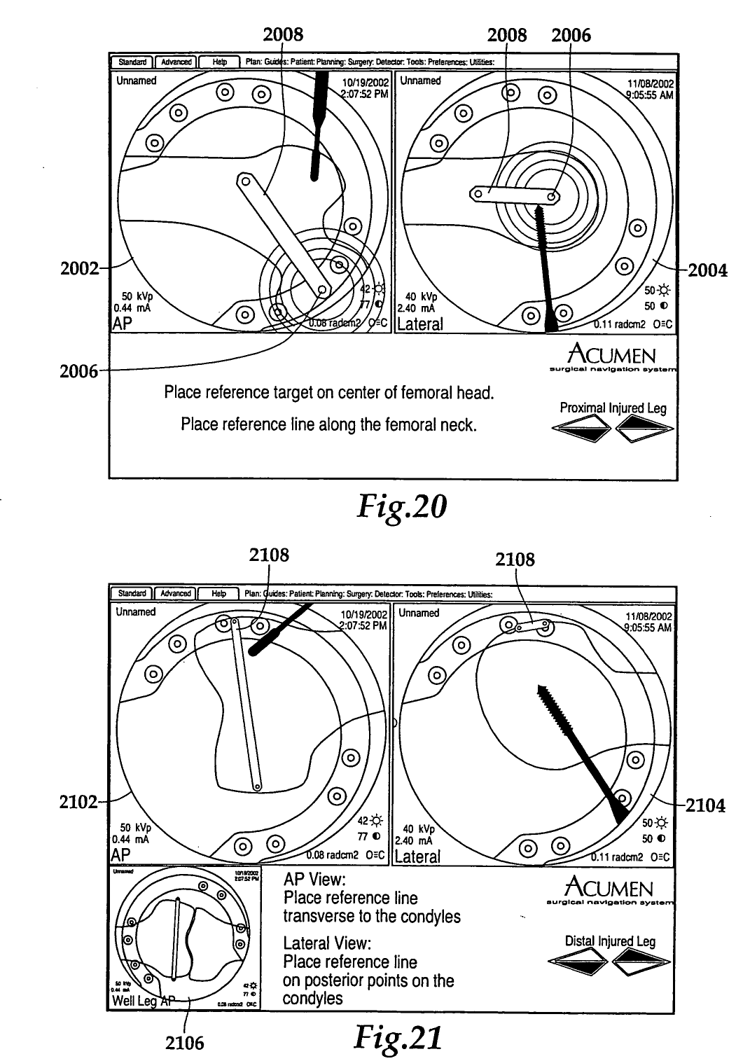

Injured-leg planning continues at steps 622 at the proximal end of the injured femur by the surgeon marking in the images the center of the femoral head and the axis of the femoral neck substantially in the same manner as discussed in connection with step 602. This information will be used to calculate reference length and version for the injured leg. FIG. 20 is a representative screen of the page displayed for this step. Its display includes the stored A/P and M/L images of the proximal end of the femur in windows 2002 and 2004. Like other pages, it includes written instructions prompting the surgeon to mark certain landmarks, namely, the femoral head and neck using a bull's eye marker 2006 and 2008, just as in FIG. 14.

In a manner similar to step 606, the same landmarks used in marking the distal end of the well femur in step 606 are marked at step 624 by the surgeon and stored for use in calculating reference length and version for comparison to the well leg. A representative screen of a display page for this step is shown in FIG. 21. It includes the stored A/P and M/L images 2102 and 2104 for the distal end of the injured leg. It also includes an A/P shot of the distal end of the well femur 2106 for reference to ensure proper marking of the landmarks on the images 2102 and 2104. The reference line is shown on image 2106 in the position marked by the surgeon at step 606. As with step 606, trans-epicondylar axis is marked on the images with reference line 2108 and stored.

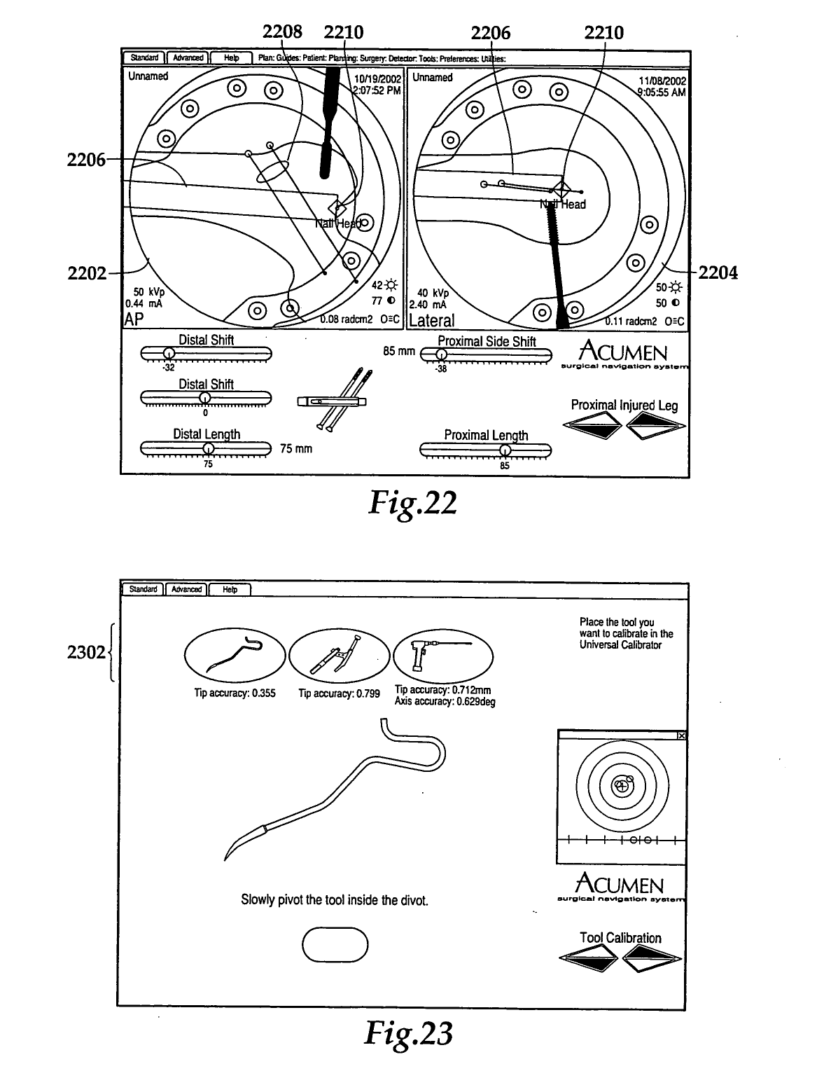

Once the reference points are marked, the process proceeds to steps 628 and 630, where the surgeon indicates to the process the entry point for the nail and desired position of the nail head and the screws that lock the nail head. As show in FIG. 22, a representative screen of an example of a page for receiving this information from the surgeon, the stored A/P and M/L images 2202 and 2204 of the distal end of the injured femur are displayed and overlaid with a representation 2206 of the previously selected nail and the locking screws 2208. The nail head 2210, which defines the entry point for the nail into the femur, is also indicated. The surgeon shifts and rotates the representation of the nail so that it fits properly in the canal and the locking screws extend up the neck of the femur shown in the images. The representations of the screws are fixed to the representation of the nail, and rotate and shift with it. When the surgeon is satisfied with the placement of the nail and locking screws, this information is stored.

As a final step before execution, tools previously selected for use in the procedure are calibrated if they are not already calibrated at step 632. A representative screen of an exemplary page that may be displayed at this step is shown in FIG. 23. A list 2302 of selected tools is displayed. A surgeon selects each tool on the list for calibration. When the tool comes into the field view of the tracking system of the CAS system, the tool is recognized and instructions for calibration are displayed. During this step, the tip and, optionally, axis of each tool is calculated with respect to a known point on a calibration fixture according to known methods. The calibration information is stored by the CAS system so that the relationship between the displayed representation of the tool and the diagnostic images is the sane as the relationship between the actual tool and the patient.

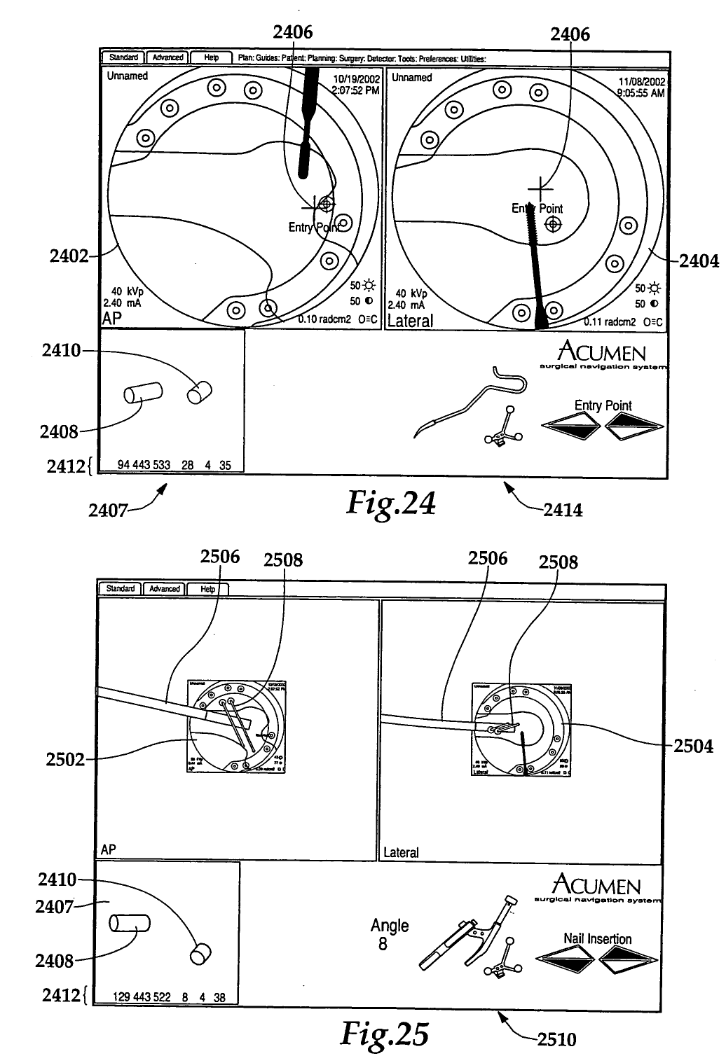

Referring now to FIG. 7, steps 702, 704, 706 and 708 involve guiding the surgeon to the correct entry point for inserting the nail. Referring now also to FIG. 24, the previously acquired and stored A/P and MIL images 2402 and 2404 are displayed. The entry point is also marked with markers 2406. Although not explicitly shown in the figures, the point of the tool selected/or use in forming the entry, in this case an awl, is continuously tracked by the CAS system and a representation of the position of the tip of the tool displayed on the images. The CAS system is continuous tracking relative changes in positions of the two fragments using trackable marker array 224 (see FIG. 2) attached to each fragment. The arrays are attached prior to registration of the images with the patient so that registration is not lost due to movement of either femoral fragment. With each leg fragment being tracked to maintain registration, the CAS system will compensate for movement of the fragments when displaying the position of tracked tools on the diagnostic images on the CAS system's display. In area or window 2414 of the display, illustrations of the objects being tracked are displayed, in this example, and awl and a trackable marker array. These illustrations also provide an indication to the surgeon if the tool or the marker array are out of the field of view of the camera by displaying, in the illustrated embodiment, a red outline on respective images in area 2414. This function is also present in subsequent tracking steps.

In window or area 2407 the relative positions and orientations of the proximal and femoral fragments of the fractured femur are indicated by representations 2410 and 2408. This window is preferably displayed during steps 708 and 714. Displayed in area 2412 is reference length and version information that is continuously calculated based on the relative positions of the fragments. This tracking is possible due to the known relationship between trackable marker array and the reference landmarks specified on the fragment. At the time when the landmarks on each fragment were specified, the positions of the trackable markers were also stored, thereby permitting the relative relationship to be determined. Using the relative relationship between each trackable marker 224 and the landmarks on the fragment to which it is attached, the referenced lengths and version are calculated based on the relative positions of the two trackable markers.

Referring now to FIGS. 7 and FIG. 25, after the surgeon forms the entry for the nail, he will “ream” the canal of the femur to prepare it for introduction of the nail. Although not tracked in the illustrated embodiment, the instrument used for reaming could be tracked to and its position could also be displayed on the images to ensure that it successfully bridges the fracture and enters the canal of the other fragment. Since the reaming device must be flexible and is located inside the femur, optical tracking cannot be used. Magnetic tracking, though less precise, could be employed. Once the canal is prepared, the surgeon will employ a tool for inserting the nail, referred to as a nail inserter. The inserter is tracked by the CAS system. Bringing the nail inserter into view of the tracking system signals the application process to move to the next step, namely, to step 710. The geometric relationship between the tool and the nail is known from the calibration step performed earlier. Therefore, by tracking the tool inserter, which remains outside the patient, the position of the nail is known.

In FIG. 25, the stored A/P and MIL images of the proximal end of the femur are displayed. Also displayed on images using representation 2506 is the current position of the nail and the screws as the nail is being inserted and rotated. The nail insertion tool is tracked. The position of the nail and screws is determined from the position of the nail insertion tool and the geometric relationship between the nail insertion tool and nail. As in FIG. 24, window 2407 displays the representations of the two fragments 2408 and 2410, of the femur in the relative positions and calculated reference lengths and versions 2412. The surgeon will use the nail and screw representations to ensure that the screws are correctly aligned with the femoral neck. The representations of the locking screws can be used as guides for drilling and inserting the screws.

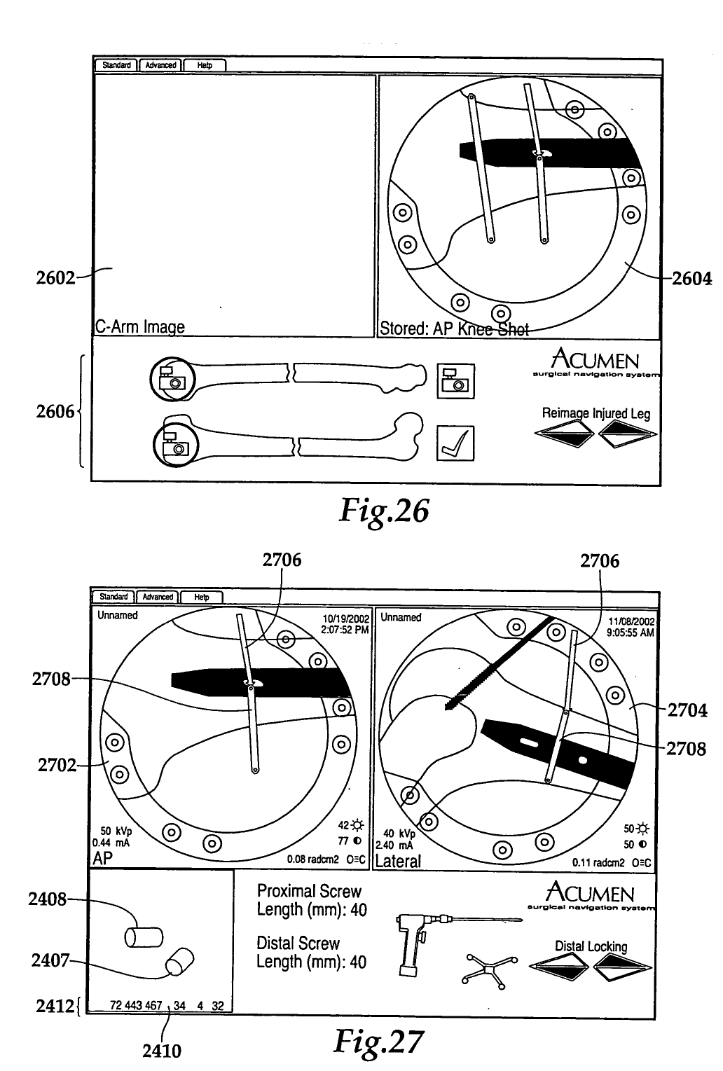

Once the surgeon inserts the nail and the proximal locking screws, the distal end locking screws must be inserted. The nail guide does not typically incorporate an external guide due at least in part to a possibility of the nail bending during insertion. In order to locate screw openings in the nail and determine trajectory of the screws, another set of lateral and A/P and M/L images of the distal end of the femur is required. Therefore, at step 712, the surgeon is prompted to acquire the additional images. FIG. 26 is an example of a page for guiding the surgeon in capturing the images. The current image for the fluoroscope is shown in window 2602. If the image is acceptable, it is stored and shown in window 2604. The shots or images to be acquired are, in this example, graphically illustrated in area 2606.

The second set of stored A/P and M/L images of the distal end of the femur should clearly show the screw holes in the distal end of the nail. In order to clearly see the holes, the lateral image needs to be a true lateral image relative to the nail. When a surgeon brings the instrument previously specified as being used for distal screw insertion into the area of focus of the tracking system, the CAS system preferably automatically displays a screen or page similar to the one of FIG. 27 and performs steps 714 and 716. The page of FIG. 27 includes the stored A/P image 2702 and lateral image 2704 of the distal end of the nail. To guide a surgeon in inserting the locking screw, a representation 2706 of the instrument being used for the insertion is superimposed on the images. A representation 2708 of the locking screw on the end of the instrument is also superimposed.

At the conclusion of the procedure, the surgeon is prompted to specify whether to archive data generated by the procedure for later reference. The CAS system archives the data as directed, such as to a disk drive or removable media. This step is not illustrated.

If desired, the different steps discussed herein may be performed in any order and/or concurrently with each other. Furthermore, if desired, one or more of the above described steps may be optional or may be combined without departing from the scope of the present invention.

Embodiments of the present invention may be implemented in software, hardware, application logic or a combination of software, hardware and application logic. The software, application logic and/or hardware may reside on processor-based system 16 or on a removable storage medium. If desired, part of the software, application logic and/or hardware may reside on processor-based system 16 and part of the software, application logic and/or hardware may reside on the removable storage medium.

What is claimed is:

1. Apparatus for assisting with surgical procedure, comprising:
a localizer;
a computer in communication with the localizer, the computer storing and executing instructions for displaying a plurality of screens, a first one of the plurality of screens corresponding to a planning step for a procedure for inserting an intramedullary nail and a second one of the plurality of screens corresponding to a navigation step of the procedure, the first one of the plurality of screens assisting with selection of the nail based on a patients anatomy and the second one of the plurality of screens indicating the position of the nail as it is being inserted into the patient's femur.
